(19) United States
(12) Patent Application Publication
(10) Pub. No.: US 2004/0121414 A1
(43) Pub. Date: Jun. 24, 2004

(54) Method for diagnosis of hepatocellular carcinoma using TGFBETA 1 as a serologic marker

(76) Inventors: Young-Hwa Chung, Seoul (KR); Byung-Cheol Song, Jeju-si (KR); Jeong A. Kim, Seoul (KR)

Correspondence Address: Michael N. Mercanti, Roberts and Mercanti, L.L.P., Suite 203, 105 Lock Street, Newark, NJ, 07103, US

(21) Appl. No.: 10/329,255

(22) Filed: Dec. 23, 2002

(51) Int. Cl. G01N 33/574

(52) U.S. Cl. 435/7.23

(57) ABSTRACT

The present invention relates to a method for diagnosis of hepatocellular carcinoma using TGF-β 1 as a serologic marker, particularly, to a method for diagnosis of hepatocellular carcinoma in which hepatocellular carcinoma can be identified by measuring the level of TGF-β 1 in plasma or serum. Since TGF-β 1 is highly sensitive as a serologic marker comparing to AFP that has been widely used as a serologic marker for the detection of hapatoma, TGF-β 1, as a serologic marker, is very useful for the accurate early detection and diagnosis of hepatocellular carcinoma.

### TECHNICAL FIELD

The present invention relates to a method for diagnosis of hepatocellular carcinoma, more precisely, to a method for diagnosis of hepatocellular carcinoma using TGFβ 1 as a serologic marker.

### BACKGROUND ART

Hepatocellular carcinoma is one of the malignant tumors. Once diagnosed as hepatocellular carcinoma, the average survival period of patients is 2-4 months and 5-year survival rate is under 20% only if treated with surgical resection. Hepatocellular carcinoma (HCC) is not the only one in the category of primary liver cancer but it takes more than 85% of all, so that it is often called “hepatoma”. In case HCC is detected as already being advanced, there is no effective treatment and its prognosis is not good, either. Thus, diagnosis at early stages of HCC is the most effective way to increase survival rate. Patients with HCC do not feel any symptom until it is already progressed to some extent. Thus, it is important to group patients who are liable to have HCC and examine them regularly to diagnose HCC in the early stage, which is the best way to detect HCC from subclinic status.

Methods for diagnosis of HCC at early stages have been developed. Serologic method to examine the level of serum alpha-fetoprotein (referred as “AFP” hereinafter) is one way (Liaw Y F et al., Gastroenteroogy, 1986, 30, 263-267; Colombo M. et al., N. Engl. J. Med., 1991, 325, 675-680; Oka H. et al., Hepatology, 1990, 12, 680-687). AFP is the first tumor-marker used to detect HCC in patients with chronic hepatic diseases. AFP protein is once contained in the blood of fetuses and later is missing in adults. However, some of HCC patients often show high level of serum AFP. Normal serum AFP level is under 20 ng/ml. When the level of serum AFP increases or is already over 400 ng/ml, the possibility of HCC is very high. Thus, 400 ng/ml has been used as a cut-off value in diagnosing HCC (Shinagawa T. et al., Gastroenterology, 1984, 86, 495-502; Chen D S. et al., Gastroenterology, 1984, 86, 1404-1409). But, as for the patients with small hepatocellular carcinoma (small HCC), defined as 2-3 cm or less in diameter, the sensitivity decreases much. For example, only 4.5-22% of patients with small HCC have been reported to have serum AFP level over 400 ng/ml and 40% of them could have normal value. Thus, measuring serum AFP levels is not accurate for the early detection and diagnosis of HCC (Chen D S et al., Gastroenterology, 1982, 83, 1109-1119; Shinagawa T. et al., Gastroenterology, 1984, 86, 495-502; Ebara M. et al., Gastroenterology, 1986, 90, 289-298). Besides, even 15-20% of patients with advanced HCC might show normal values of serum AFP. And nonspecific elevation of serum AFP has been frequently found in patients with acute hepatitis, chronic hepatitis and liver cirrhosis (Silver H K et al., Cancer Res, 1974, 34, 244-247; Di Bisceglie A M et al., Cancer, 1989, 64, 2117-2120; Taketa K, Hepatology, 1990, 12, 1420-1432). Such non-specific increase of serum AFP makes the cut-off value of serum AFP higher, consequently, the sensitivity is further decreased. In order to supplement the insensitivity, lectin reactive AFP has been used (Taketa K. et al., Cancer Res, 1993, 53, 5419-5423; Shiraki K. et al., Hepatology, 1995, 22, 802-107), or isoeletric focusing (IEF) assay has been performed. However, those methods are limited in use because of troublesome procedures and high expense.

Ultrasonography is also useful for diagnosis of HCC at early stages. However, it is not only difficult with that method to identify whether it is HCC or other benign tumors but also hard to diagnose HCC accurately and objectively because ultrasonic permeability is very low in patients with serious liver cirrhosis. Another method for diagnosis of HCC is using prothrombin induced by Vitamin K absence or Antagonist Π (PIVKA-Π) (Chan C Y et al., J Hepatol., 1991, 13, 21-24; Weitz I C et al., Hepatology, 1993, 18, 990-997). This method has been widely performed in Japan recently. This method is to measure the value of an inactive prothrombin (Des-g-carboxy prothrombin; referred as “DCP” hereinafter) produced by post-translational defect of carboxylase system in hepatoma cells. However, the sensitivity of DCP, as a marker, is about 50-60% in patients with HCC and 15-30% in patients with small HCC. Not only the low sensitivity but also high cost have been pointed out for this method.

To diagnose HCC at early stages, serum AFP test and ultrasonography have been performed together so far. But it is still required to develop new tumor-markers that have sufficient sensitivities as well as specificities in detecting HCC at early stages.

The present inventors have confirmed earlier that TGFβ 1 mRNA was over-expressed in HCC tissues, especially in small-sized and well-differentiated HCC tissues comparing to surrounding liver tissues (Song B C et al., Kor J. Gastroenterol., 1999, 34, 774-783).

Thus, the present inventors have accomplished this invention by confirming that small HCC is effectively diagnosed by measuring the level of plasma TGFβ 1 over-expressed in patients with small HCC.

### DISCLOSURE OF THE INVENTION

Transforming growth factory-β 1 (TGFβ 1) is a multifunctional cytokine involved in the regulation of growth and differentiation of both normal and transformed cells (Blobe G C et al., N. Engl. J. Med., 2000, 342, 1350-1358), and plays an important role in hepatic fibrosis, cell cycle arrest and apoptosis. TGFβ 1 is generated as a latent precursor (preproprotein) in platelets, endothelial cells and inflammatory cells, and is increased by internal and external stimuli. Secreted TGFβ 1 ought to be separated as it still has C-terminal by stimuli such as acids, bases, heats, urea and proteases in order for precursor to transmit signals by attaching to receptors of target cell membranes and transformed to an active form by forming homodimer or heterodimer by disulfide bond. The disclosure of some parts of signal transduction system of apoptosis by TGFβ 1 triggered the studies on the role of this cytokine in the development of HCC. TGFβ 1 mRNA and protein were over-expressed in HCC tissues and plasma TGFβ 1 was increased in patients with HCC (Kim H G et al., Kor J. Intern. Med., 2000, 15, 165-170; Abou-Shady M. et al., Am. J. Surg., 1999, 177, 209-215; Factor V M et al., Cancer Res., 1997, 57, 2089-2095).

The present inventors confirmed earlier that TGFβ 1 mRNA was over-expressed in HCC tissues, especially in small-sized and well-differentiated HCC tissues (Song B C et al., Kor J. Gastroenterol., 1999, 34, 774-783). In addition, the present inventors confirmed in previous studies that TGFβ 1 was much increased in plasma of patients with small HCC, compared with patients with liver cirrhosis. Thus, the present inventors have expected plasma TGFβ 1 to be effectively used as a serologic marker for the diagnosis of small HCC. The present invention is characterized by using TGFβ 1 as a tumor-marker for the detection or the diagnosis of HCC at early stages.

The present invention provides a method for diagnosing HCC by measuring the level of TGFβ 1 in biological samples taken from examinees.

The method for diagnosing HCC of the present invention comprises the following steps and is characterized by the same:

(a) Taking biological samples from examinees;

(b) Measuring the level of TGFβ 1 in the above samples; and

(c) Positively identifying HCC with TGFβ 1 level over 600 pg/ml, and diagnosing HCC with TGFβ 1 level over 800 pg/ml.

In the preferred embodiments of the present invention, serum and plasma were used as biological samples. Before measuring, it was recommended to activate latent TGFβ 1 in serum or in plasma to immunoreactive TGFβ 1. To activate latent TGFβ 1 to immunoreactive TGFβ 1, the mixture consisting of 3.75 M acetic acid and 15 M urea was reacted with plasma. At this time, the final pH of the reaction mixture was adjusted to 2.0-2.5.

To measure the level of TGFβ 1 in serum or in plasma, commonly known methods for measuring the amount of protein can be used. Particularly, for instance, prepared an antibody binding to TGFβ 1 and reacted thereof with plasma sample taken from examinee in order to induce antigen-antibody reaction, resulting in the measurement of TGFβ 1 level. And one or more methods selected from a group consisting of immunoblotting, ELISA, enzyme-linked immunoassay (EIA) and radioimmunoassay (RIA) can be used for the measurement of the level.

The method for measuring the level of TGFβ 1 in plasma using immunoblotting comprises the following steps:

1) Absorbing antibody against TGFβ 1 to matrix;

2) Adding plasmas of examinees to antigen-absorbed membrane for reaction and then washing thereof;

3) Adding coloring enzyme or fluorescent material-conjugated antibody to the above membrane and reacting thereof; and

4) After inducing color development by adding coloring agent to the above membrane, observing the specificity of antigen-antibody reaction.

For the matrix of the above step 1), nitrocellulose membrane, 96-well plate synthesized by polyvinyl, 96-well plate synthesized by polystyrene and slide glass can be used.

For the coloring enzyme conjugated to antibody of the above step 3), peroxidase, alkaline phosphatase, etc can be used and FITC, RTTC, etc can be used as fluorescent material.

For the coloring agent of the above step 4), 4-chloro-1-naphtol (4CN), Diaminobenzidine (DAB) and Aminoethyl carbazole (AEC) can be used.

The method for measuring the level of TGFβ 1 in plasma of examinees using ELISA comprises the following steps:

1) Absorbing antibody against TGFβ 1 on well plate;

2) Adding plasma samples to the above wells for reaction and then washing thereof;

3) After washing the above well plate, adding the secondary antibody conjugated with coloring enzyme or fluorescent material thereto and reacting thereof; and

4) After inducing color-development by adding coloring agent, measuring OD with ELISA reader.

The level of plasma TGFβ 1 in examinees can be detected by using biological microchip on which antibody against TGFβ 1 is fixed. And mass-analysis of samples is possible by using known biological microchip and automatic microarray system along with ELISA.

TGFβ 1 was preferably measured using an ELISA kit (R&D systems, Minneapolis, Minn.) for human TGFβ 1 according to the manufacturer's instruction. All tests were performed a couple of times. The cut-off value of TGFβ 1 is 600 pg/ml for identifying HCC at early stages, which is small HCC smaller than 3 cm in diameter and 800 pg/ml for the diagnosis of HCC.

The present invention also provides a diagnostic kit for HCC characterized by having TGFβ 1-specific antibody and serologic assay components.

The above TGFβ 1-specific antibody can be prepared by conventional methods. The diagnostic kit of the present invention may include buffer solution, antigen protein, standard antibody, coloring enzyme or fluorescent material-conjugated secondary antibody and coloring substrate.

To diagnose HCC with the kit, automatic analysis system using a biological microchip is also used. For instance, ELISA is performed using a glass slide chip on which TGFβ 1-specific antibody is coated. The diagnostic kit includes a biological microchip on which antigen protein is fixed, buffer solution, standard antibody and the secondary antibody.

To investigate efficiency of TGFβ 1 in diagnosing small HCC, TGFβ 1 and AFP that has been widely used as a marker for diagnosis of HCC were taken from 38 patients with small HCC (smaller than 3 cm in diameter), 31 patients with liver cirrhosis and 23 normal people. Plasmas and sera were taken from those and the level of TGFβ 1 in plasma and the level of AFP in serum were measured. As a result, plasma TGFβ 1 level of small HCC patients was remarkably higher than that of normal control group (see Table 1), suggesting that the sensitivity of TGFβ 1, as a serologic marker, is sufficient. In spite of non-specific increase of plasma TGFβ 1 in patients with liver cirrhosis, overlapping range was narrower than that of AFP (see FIG. 1). The cut-off values were determined and diagnostic performances of plasma TGFβ 1 and serum AFP were compared using receiver operating characteristic (ROC) curve (see FIG. 2). At the cut-off level 800 pg/ml of plasma TGFβ 1, the sensitivity was 68%, which was much higher than that of serum AFP (24%) (see Table 2). This result suggests that TGFβ 1 is a superior tumor-marker to AFP in diagnosis of HCC at early stages.

The present inventors determined the cut-off values of plasma TGFβ 1 to diagnose small HCC to be 800 pg/ml since the specificity is over 95% at the value. Although the best cut-off value of plasma TGFβ 1 for diagnosis was 600 pg/ml according to ROC curve, the specificity was down to 81%. The sensitivity was elevated to 87% at that level, though. Generally the cut-off values with high sensitivity are useful for screening-HCC, only if the values have sufficient specificity. On the contrary, the values with high specificity, if sensitivity is not much sacrificed, are useful for confirming a disease (Griner P F et al., Ann. Intern. Med., 1981, 94, 555-600). Therefore, the cut-off values of plasma TGFβ 1 ought to be 600 pg/ml for screening small HCC and 800 pg/ml for diagnosis thereof.

### BRIEF DESCRIPTION OF THE DRAWINGS

FIG. 1 is a set of graphs showing the distribution of serum AFP (A) and plasma TGFβ 1 (B). The values of plasma TGFβ 1 and serum AFP are presented in a logarithmic scale,

FIG. 2 is a graph showing the receiver operating characteristic (ROC) curve prepared by measured levels of plasma TGFβ 1 and serum AFP.

### BEST MODE FOR CARRYING OUT THE INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Patients Selection

Among 234 HCC patients diagnosed at the Asan Medical Center for a year (from May 1988 to April 1999), 42 patients (17.9%) had small HCC. Plasma samples were available from 38 of those 42 patients. Hepatocellular carcinomas were diagnosed clinically in patients with hypervascular mass in the liver and serum AFP levels exceeding 400 ng/ml (n=8) or through histologic means (n=30). The cirrhosis controls were randomly selected on the basis of the following criteria: clinically relevant portal hypertension (presence of esophageal varices and/or ascites, splenomegaly with platelet count <100,000/ [symbol] ³) and imaging feature suggestive of liver cirrhosis in (Bruix J et al., Gastroenterology, 1996, 111, 1018-22; Di Lelio A et al., Radiology, 1989, 172, 389-92). To measure the normal plasma level of TGFβ 1, 23 normal volunteers were also investigated. None had any documented organic diseases, and all were negative for hepatitis B surface antigen and hepatitis C virus. The baseline characteristics of patients with small HCC and liver cirrhosis controls are presented in Table 1.

TABLE 1

| | | Patients with | Patients with |
|---|---|---|---|
| Variables | | small HCC (n = 38) | liver cirrhosis (n = 31) |
| Age, | | 58(33-76) | 52(32-75) |
| Median (range) | | | |
| Gender (M/F) | | 33/5 | 28/3 |
| Etiology | | 33/4/1 | 23/5/3 |
| (HBV/HCV/alcohol) | | | |
| Child-pugh class | | 31/6/1 | 24/7/0 |
| (A/B/C) | | | |
| serum | <20 | 20(52.6) | 23(74.2) |
| AFP | 20-200 | 9(23.7) | 7(22.6) |
| (ng/ml) | 201-400 | 1(2.6) | 1(3.2) |
| | >400 | 8(21.1) | 0(0) |

| | |
|---|---|
| Size, | 2(0.8-3) |
| Median (range) | |

HCC: hepatocellular carcinoma,
HBV: hepatitis B virus,

HCV: hepatitis C virus, AFP: alpha-fetoprotein

As shown in Table 1, the ages of the patients with small HCC ranged from 38 to 76 years (median: 58 years), which was older than the ages of the liver cirrhosis patients (p=0.01). Serum AFP levels were frequently elevated in patients with small HCC compared with liver cirrhosis patients (P=0.02). Serum AFP was elevated (>20 ng/ml) in 47.4% (18 out of 38) of patients with small HCC and 25.8% (8 out of 31) of those with liver cirrhosis. The number of HCC nodules was 1 nodule in 31 patients, 2 nodules in 4 patients, and 3 nodules in 3 patients. The median size of the HCCs was 2.0 cm in diameter, ranging from 0.9 to 3.0 cm. Ultrasonography was performed at 3-6 month intervals for a follow-up period of 12 months or more to determine the presence or absence of intrahepatic masses, which were not found in liver cirrhosis control patients. There were no significant differences between patients with small HCC and liver cirrhosis controls in gender, etiology of liver diseases, and Child-Pugh class.

### EXAMPLE 2

### Measurement of Plasma TGFβ 1

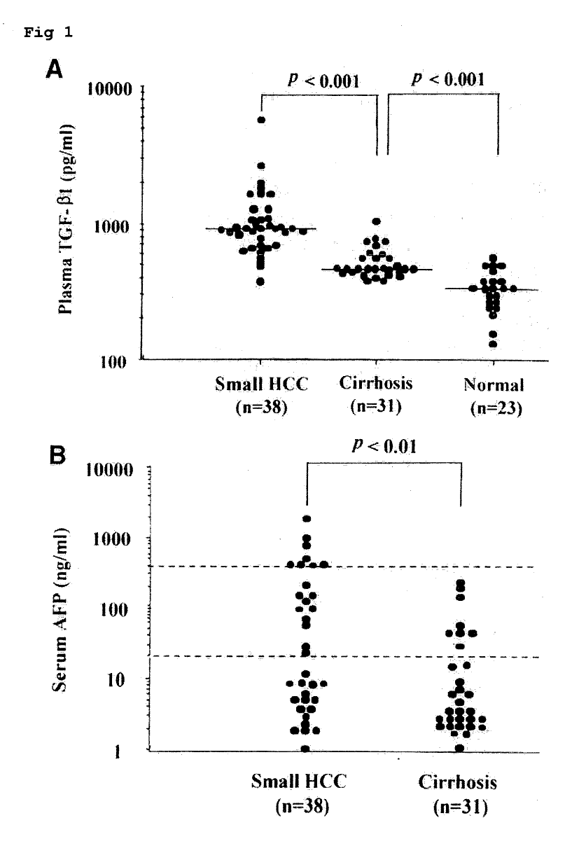

From the patients with small HCC and liver cirrhosis of the above Example 1, venous blood was collected in heparinized tubes and immediately centrifuged (10,000×g) at 4° C. for 20 minutes. Following centrifugation, the present inventors collected plasma cautiously to avoid touching the buffy coat. The plasma samples were kept at −20° C. until used for assay. To activate latent TGFβ 1 to immunoreactive TGFβ 1, 50 μl of the mixture consisting of 3.75 M acetic acid and 15 M urea was added to a tube containing 100 μl of plasma and incubated at room temperature for 10 minutes. The final pH of the reaction mixture was adjusted to 2.0˜2.5, which has been reported to activate the latent TGFβ 1 completely to the active form of TGFβ 1 (Brown P D et al., Radiology, 1989, 172, 382-392). Acidified samples were neutralized by adding 50 μl of mixture consisting of 4 M NaOH and 1.5 M HEPES. After then, the samples were serially diluted with phosphate-buffered saline for enzyme linked immunosorbent assay (ELISA). TGFβ 1 in the samples was measured using an ELISA kit for human TGFβ 1 (R&D systems, Minneapolis, Minn.) according to the manufacture's instructions. All tests were performed in duplicate. The levels of TGFβ 1 were calculated by averaging the results and represented in FIG. 1A.

As a result, the coefficients of variation of intra-assay and inter-assay were less than 10% for all measurement (FIG. 1A).

### EXAMPLE 3

### Measurement of Serum AFP

From the patients with small HCC and liver cirrhosis of the above Example 1, serum was collected according to the conventional method (Chayvialle J A P et al., Dig Dis, 1974, 19, 1102-1110). Serum AFP levels were determined using a commercially available radioimmunoassay kit (Abbott Laboratories, North Chicago, Ill.). All tests were performed in duplicate. The levels of AFP were calculated by averaging the results and represented in FIG. 1B. The coefficients of variation of intra-assay and inter-assay were less than 10% for all measurement (FIG. 1B).

### EXAMPLE 4

### Comparison of Plasma TGFβ 1 and Serum AFP Levels in Patients with Small HCC and Liver Cirrhosis

The present inventors prepared a distribution chart by statistical analysis of plasma TGFβ 1 and serum AFP levels in patients with small HCC and liver cirrhosis measured in the above Example 2 and Example 3. Particularly, the levels of plasma TGFβ 1 and serum AFP were expressed as mean± standard deviation (range). The differences between dichotomous variables were analyzed by Fisher exact test or chi-square test. For the continuous variable, the Student t-test (when the data showed normal distribution) or Mann-Whitney U test (when the data did not show normal distribution) was used. Receiver operating characteristic (ROC) curve was used to determine the cut-off values and compare the diagnostic performance of plasma TGFβ 1 and serum AFP (Zweig M H et al., Clin Chem., 1993, 38, 561-577; Hanley J A et al., Radiology, 1983, 148, 839-843). A p-value of less than 0.05 (two-tailed) was considered to be statistically significant.

As a result, the plasma levels of TGFβ 1 were significantly higher in patients with small HCC (2.98±0.22 pg/ml) than those in patients with liver cirrhosis (2.69±0.11 pg/ml) (log value, pg/ml; 2.98±0.22 vs. 2.69±0.11).

Besides, normal serum AFP level was 20 ng/ml or less. And serum AFP was elevated in only 47% (18 out of 38) of patients with small HCC. It means that the serum AFP is not very useful for the diagnosis of hepatocellular carcinoma.

### EXAMPLE 5

### Sensitivities and Specificities of Various Plasma TGFβ 1 and Serum AFP Levels in the Diagnosis of Small HCC

The present inventors calculated the sensitivities and specificities of plasma TGFβ 1 and serum AFP levels in diagnosing small HCC by the same statistical analysis of the above Example 4.

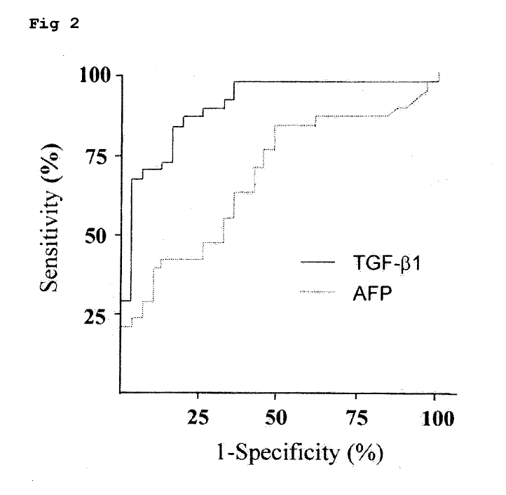

As a result, the calculated area under the ROC curve was 0.9 for plasma TGFβ 1 and 0.65 for serum AFP. There was a significant difference between the two areas (FIG. 2). The area under the ROC curve of plasma TGFβ 1 was significantly greater than that of serum AFP, indicating that plasma TGFβ 1 is superior to serum AFP in diagnosing small HCC.

Sensitivities and specificities at several values of plasma TGFβ 1 and serum AFP are summarized in Table 2.

TABLE 2

| | | |
|---|---|---|
| Cut-off | Sensitivity (%) | Specificity (%) |

| | | | |
|---|---|---|---|
| AFP | 20 | 47.4 | 74.2 |
| (ng/ml) | 100 | 31.6 | 90. 3 |
| | 200 | 23.7 | 96.8 |
| | 400 | 21.1 | 100 |
| TGFβ 1 | 500 | 92.1 | 67.7 |
| (pg/ml) | 600 | 86.8 | 80.6 |
| | 800 | 68.4 | 96.8 |
| | 1,000 | 31.6 | 96.8 |

As shown in Table 2, keeping in mind a sufficient degree of specificity in the clinical diagnosis of cancer, the cut-off values of plasma TGFβ 1 and serum AFP were 800 pg/ml and 200 ng/ml, respectively, where the specificities were 95%. At the cut-off level of plasma TGFβ 1, the sensitivity was 68%. However, the sensitivity of serum AFP was only 24% at the cut-off level of 200 ng/ml, where the acceptable range of specificity for cancer diagnosis is over 95%. The current data were quite similar to the sensitivities of 9-29% at the same level of serum AFP reported (Chen D S et al., Gastroenterology, 1982, 83, 1109-1119; Ebara M. et al., Gastroenterology, 1986, 90, 289-298; Taketa K., Hepatology., 1990, 12, 1420-1432).

Therefore, TGFβ 1 can be a useful serologic marker in detecting HCCs earlier because it shows higher sensitivity than AFP with the same level of specificity in the diagnosis of small HCCs.

### INDUSTRIAL APPLICABILITY

As described hereinbefore, TGFβ 1 can be effectively used for the diagnosis of small HCCs since it is highly sensitive as a serologic marker comparing to AFP that has been widely used as a serologic marker for the detection of HCCs.

What is claimed is:

1. A method for diagnosis of hepatocellular carcinoma using TGF-β1 as a serologic marker.

2. The method for diagnosis of hepatocellular carcinoma as set forth in claim 1, wherein the method is comprised of the following steps and is characterized by the same:
(a) Taking biological samples from examinees;
(b) Measuring the levels of TGFβ 1 in the above samples; and
(c) Positively identifying HCC with TGFβ 1 level over 600 pg/ml, and diagnosing HCC with TGFβ 1 level over 800 pg/ml.

3. The method for diagnosis of hepatocellular carcinoma as set forth in claim 2, wherein the hepatocellular carcinoma is small hepatocellular carcinoma (small HCC).

4. The method for diagnosis of hepatocellular carcinoma as set forth in claim 2, wherein the biological sample of step (a) is serum or plasma.

5. The method for diagnosis of hepatocellular carcinoma as set forth in claim 2, wherein the activating step of latent TGFβ 1 to immunoreactive TGFβ 1 can be added before the step (b).

6. The method for diagnosis of hepatocellular carcinoma as set forth in claim 5, wherein the measurement of TGFβ 1 in step (b) is performed by one or more methods selected from a group consisting of ELISA, EIA, RIA and immunoblot.

7. A diagnostic kit for hepatocellular carcinoma comprising specific antibody to TGFβ 1 and serologic assay components.
